(19) United States
(12) Patent Application Publication
(10) Pub. No.: US 2022/0347019 A1
(43) Pub. Date: Nov. 3, 2022

(54) COMPRESSION BANDAGE AND COMPRESSION BANDAGE COMBINATION

(71) Applicant: KOB GMBH, Wolfstein (DE)

(72) Inventors: Ferdinand Tamoué, Ludwigshafen am Rhein (DE); Günter Langen, Wolfstein (DE)

(21) Appl. No.: 17/620,892

(22) PCT Filed: Jun. 22, 2020
(86) PCT No.: PCT/EP2020/067267

§ 371 Date: Dec. 20, 2021

(30) Foreign Application Priority Data
Jun. 21, 2019 (DE) 10 2019 116 825.2

(51) Int. Cl. A61F 13/08; D04H 3/009; D04H 3/011; D04H 3/16; D04H 3/007; D04H 1/02; D04H 1/4258; D04H 1/52; D04H 1/4334; D04H 1/435; D04H 1/4291; D04B 21/16; D04B 21/18

(52) U.S. Cl. CPC A61F 13/08; D04H 3/009; D04H 3/011; D04H 3/16; D04H 3/007; D04H 1/02; D04H 1/4258; D04H 1/52; D04H 1/4334; D04H 1/435; D04H 1/4291; D04B 21/165; D04B 21/18; D10B 2509/028; D10B 2401/061

(57) ABSTRACT

The invention relates to a compression bandage comprising a nonwoven-based bandage layer that has been stitched over with elastic textile threads by stitch bonding methods, the textile threads being heat-shrinkable and not elastomeric and the compression bandage having an elasticity after heat shrinking of the textile threads by 50-200%, particularly 50% to 90% and especially 50% to 70%. The invention also relates to a compression bandage combination.

The invention relates to a compression bandage and to a compression bandage combination comprising two layers.

Compression bandages are used in the prior art for diabetic ulcers, for example. A problem here is that the clinical picture means that different challenges inevitably have to be addressed, namely providing adequate compression on the one hand and a padding effect on the limb to be treated on the other.

Thus, it may for example be the case that padding is applied and a compression bandage laid over this. Beyond this, there is however a consensus view that concomitant arterial occlusive disease is a contraindication.

In compression therapy, there is a fundamental distinction between the “working pressure” and the “resting pressure”, the resting pressure being the pressure exerted on the limb by the compression device, in this case the compression agent, when the limb is at rest. The working pressure is then the pressure exerted on the limb when the muscles are being moved. The working pressure should preferably be 20 to 40 mmHg above the resting pressure.

The use of different bandage types is likewise known. For instance, so-called long-stretch bandages are known, which exhibit very high elasticity and often have an extensibility of more than 200%, whereas by contrast short-stretch bandages are known that have only low extensibility and only low recoil force, but even at a very early stage do not allow any further extension and are thus capable of building up a comparatively high working pressure. On the other hand, short-stretch bandages build up only low resistance over a comparatively long span, so as to then place a very high limit on extensibility. Traditional short-stretch bandage compression therapy is a measure used to treat venous disorders. The materials for short-stretch bandages are generally produced from non-elastic materials and elasticized through finishing processes. The elasticity is however significantly reduced during the treatment. This can lead to a reduction in compression pressure in use.

Known compression bandages are described for example in EP 2 275 062 A2, which describes an inner, skin-facing elastic bandage having an elongated elastic substrate and an elongated foam layer arranged on the skin-facing side of the substrate, and a further elongated self-adhesive elastic bandage applied over this.

In addition, DE 20 2012 000 529 U1 discloses a support, conforming or compression dressing, the dressing here having at least four layers and a tensioning layer that generates the recoil force and has perforations, the remaining layers being fixed to one another through the perforations. As a consequence of the four layers provided, the dressing is comparatively costly and complex.

Reference should additionally be made to WO 2014/131976 A2, which likewise relates to an elastic bandage, comprising a non-elastic layer that encloses the elastic band and is joined herewith.

DE 10 2015 226 706 A1 discloses a compression dressing having a padding layer and a support layer that are joined to one another by means of stitch-bonding processes, over which a second compression dressing can be applied.

It is desirable to provide a compression bandage, which can alternatively also be referred to as a compression dressing, and a compression bandage combination that have high security of attachment while at the same time having good therapeutic properties.

Where the terms layer or ply are used, they refer to the same thing.

Starting out from this prior art, the invention achieves the object by means of a compression bandage having the features of claim 1 and also a compression bandage combination of claim 9.

The compression bandage comprises a nonwoven-based bandage layer overstitched by elastic textile threads by means of a stitch-bonding process, wherein the textile threads are heat-shrinkable and non-elastomeric and wherein the compression bandage has elastic extensibility after the textile threads have been heat-shrunk by 50-200%, the heat-shrinkability of the textile threads preferably being 50-90% and the heat shrinkability more preferably being 50 to 70%. As a consequence of the heat treatment, the heat-shrinkable textile threads that had been processed into the nonwoven by means of the stitch-bonding process undergo shrinkage and shortening. This also results in a shortening of the overall nonwoven construction, thereby preserving its recoil capacity and stretch-elastic properties. The shrinkage occurs preferably in the longitudinal direction. The heat-shrink treatment of the stitch-bonded nonwoven is preferably carried out with hot air (130° C. to 200° C.) or steam (saturated steam or pressurized steam at 100° C. to 140° C.)

The compression bandage is preferably a bandage having recoil values of less than 90% (DIN 61632).

In a first exemplary embodiment, the bandage layer may consist of a chemically, thermally and/or mechanically consolidated nonwoven fabric and more particularly be a thermal bond nonwoven.

The base nonwoven may preferably be a fibrous nonwoven (staple fiber nonwoven) composed of cotton, wool, viscose, polyamide, polyester, acrylic, polyolefin or mixtures of said fibers, more particularly it may be formed therefrom, these nonwovens being more particularly mechanically or chemically bonded. Alternatively, a spunbond composed of polyamide, polyester, polyolefin, acrylic or mixtures thereof may preferably be used.

The heat-shrinkable textile threads may preferably be textured multifilament yarns, bicomponent polymer fibers, and microfibers. More particularly, the textile threads may be textured polyamide yarns and/or textured polyester yarns. The stitching threads may more particularly be 78 dtex to 320 dtex textured multifilament yarn composed of polyamide, polyester, polyethylene phthalate or polybutylene phthalate or mixtures thereof. More particularly, polyamide 6 to 6.6 may be used.

It is possible for the nonwoven fabric itself, not including the heat-shrinkable textile threads, to be non-elastic or stretch-elastic.

The compression bandage may preferably be cohesive in design.

The basis weight of the bandage layer is 20 to 40 g/m², it being possible to use both white and colored bandage layers.

The bandage layer may be smooth or open-pored or perforated or embossed.

A stitching thread density of 74 to 96 threads per cm bandage width is used for overstitching. The preferred stitching thread stitch length is 2 to 5 mm.

Both open and closed fringes or tricot bindings or combinations thereof can be considered for the stitching thread bindings. The compression bandage has a basis weight of 25 to 28 g/m² (stretched) without cohesive coating and of 30 to 100 g/m² (stretched) in the cohesively coated form.

The extensibility as per DIN 61632 at a force of 3 N/cm gives rise to an extensibility of 40 to 100% in the longitudinal direction (MD) and 0 to 50% in the transverse direction (CD).

The invention also comprises a compression bandage combination comprising a first, inner bandage and a second, outer bandage, wherein the second outer bandage when used can be applied over the first, inner bandage.

Multi-ply dressings (multilayer dressings) of this kind for use as a compression dressing on the human or animal body are known. The dressings are created by successively wrapping the body part with at least two separate, different bandages. In this case it is known practice to design the inner bandage as a padded bandage that is wrapped directly onto the skin of the body part as an inner layer and over it to place a compression bandage, which is wrapped over and contiguously to the first, inner bandage as an outer layer, with the two layers adhering to one another, forming a non-slip join.

The first, inner bandage comprises a preferably first section (L1) that has a padding layer at least on its side facing the skin of a wearer and more particularly has a cohesively adhering second section (L2), the second, outer bandage being a compression bandage of the type described above. The compression bandage combination is more particularly a two-layer compression dressing having a fixed compression pressure, comprising a first bandage (component A: padding bandage) and a second bandage (component B: compression bandage of the invention), wherein component A is first applied to the body part and then component B is wrapped over it and this combination of layer A and B exerts a fixed compression pressure on the body part. More particularly, the present invention comprises a compression bandage for the treatment of venous disorders such as chronic venous insufficiency or chronic venous ulcers, but also disorders with arterial involvement such as peripheral arterial occlusive disease (PAOD) or so-called mixed ulcers.

The invention thereby solves the further technical problem of a medical multicomponent compression dressing comprising two separate elastic short-stretch bandages having limited and defined therapeutic pressure. As a result of its lower recoil force and consequently limited contact pressure, the combination of the components permits the creation of a starting material, the primary function of which is a compression bandage for the treatment of peripheral arterial occlusive disease.

Compression therapy of the lower legs is one of the cornerstones of therapy for the treatment of chronic venous insufficiency (CVI) up to and including venous leg ulcers (VLUs). In many reviews and guidelines, the effectiveness of this therapy is attested to by level 1 evidence for ulcer healing and also for recurrence prophylaxis. At the same time, there is however evidence in this literature suggesting that peripheral arterial occlusive disease (PAOD) should be considered a relative contraindication and advanced PAOD an absolute contraindication. Some authors put this information in more concrete terms, with an absolute contraindication at a Doppler index (ABI) of <0.6 or even <0.8. However, observational studies show no complications and good tolerability for moderate compression (30 mmHg) in patients with leg ulcers and PAOD (ABI 0.5-0.8) over 14 days. In CVI with leg ulcers and concomitantly diagnosed PAOD (ABI 0.5-0.8), arterial perfusion is unimpaired by the short-stretch compression applied, whereas reduced venous pump function is improved, especially when walking. Problem-free treatment of VLUs with compression of <40 mmHg is possible despite the presence of PAOD (ABI 0.5-0.8), although healing is delayed. In arteriovenous ulcers (ABI >0.6), short-stretch compression up to 40 mmHg leads to an improvement in arterial flow and in venous pump function. In summary, problem-free compression therapy seems to be possible even in the presence of concomitant PAOD up to an ABI (limit) value of approx. 0.5. A compression bandage and compression bandage combination of the invention is able to ensure a maximum compression pressure of less than 40 mmHg, even at maximum extension.

The invention thus also comprises the use of a compression bandage of the invention for the creation of a compression dressing or of a compression bandage combination for the treatment of venous disorders, chronic venous insufficiency, and venous leg ulcers even with concomitant peripheral arterial occlusive disease.

The two sections (L1, L2) of the first, inner bandage are joined to one another and adjoin one another in the longitudinal direction of the bandage or completely or partially cover one another.

In addition, the first bandage may comprise a first, padding layer and a second, support layer, the two layers being joined to one another in the unstretched state by means of a stitch-bonding process via an elastic stitching thread, the stitch length being 1.5 to 3 mm/rev with a stitching thread tension of not more than 4 cN. The elasticity may be adjusted by incorporating elastic threads, preferably rubber or polyurethane threads. For example, the elasticity can be obtained by overstitching a rigid single-layer nonwoven fabric with permanently elastic elastane threads in the longitudinal direction using the Maliwatt stitch-bonding technique. The Maliwatt stitch-bonding technique is described in Malimo Nahwirktechnologie [Malimo stitch-bonding technology], Ploch, Böttcher, Scharch, VEB Fachbuchverlag Leipzig, 1978, 1st edition. The same technique can also be used to stitch together two nonwoven fabric sheets placed on top of each other (fabric 1: standard nonwoven; fabric 2: fleece-like nonwoven wadding) to form an elastic composite nonwoven material having a fleece-like structure.

The advantage of a stitch-bonding process is that a join may be made in several places at the same time and, once joined, the two layers can no longer be separated from one another. Through the selection of the stitch length in the longitudinal direction of the fabric from which the compression bandage or the inner bandage are then produced, stitch length being understood as meaning the distance in the longitudinal direction of the stitch between two stitches, and the stitching thread tension, it is possible to adjust the elasticity of the elastic composite so that an elastic composite of the two layers that can no longer be separated by hand but is nevertheless controllable is obtained. Depending on the selection of these parameters, the finished fabric contracts on tension release, with wrinkles raised in the material.

The stitching technique and stitch length of the stitching thread for the inner bandage are regulated such that the fibers on the padding layer on one side of the composite of the two layers have skin comfort and compensation functions and that accordingly the inner bandage ultimately has two recognizably different sides that are highly functional for the pressure compensation. In addition, the stitch length must be set such that the desired absorbent and skin-friendly properties of the padding layer facing the skin are preserved.

The padding layer is the limb-facing side of the inner bandage and the support layer is the second side applied thereon.

The Malimo/Maliwatt process for joining the layers of the inner bandage and for producing the inner bandage or the compression bandage is employed here, as is known in the prior art. For example, the elasticity can be obtained by overstitching a rigid nonwoven or fabric with permanently elastic elastane threads in the longitudinal direction using the Maliwatt or Malimo stitch-bonding technique. The Maliwatt/Malimo stitch-bonding technique is described in Malimo Nahwirktechnologie [Malimo stitch-bonding technology], Ploch, Böttcher, Scharch, VEB Fachbuchverlag Leipzig, 1978, 1st edition.

The material of the inner bandage and/or of the outer bandage as well as of one or both of the bandage layers (if the bandages are multilayered) themselves may inter alia be non-elastic. This allows the elasticity that is then provided by the overstitching to be set particularly precisely. The elasticity may be present in the longitudinal and/or transverse direction, preferably in the longitudinal direction of the bandage.

Through this design, it is also possible in multilayer bandages for the two layers to be joined by means of the stitch-bonding process and here preferably a Malimo/Maliwatt method, the layers being joined in the unstretched state by means of the elastic stitching thread.

The compression bandage (outer bandage) and/or the inner bandage and, if provided, both layers of the inner bandage, i.e. the padding layer and the support layer, may preferably be non-elastic in design and elasticized only by the stitch-bonding process.

In addition, it may be the case for the inner bandage that at least one of the two layers—the padding layer and the support layer—are composed of a nonwoven material. More particularly, the fabric with a fleece-like structure may consist of a single-layer or multilayer wadding-like nonwoven fabric.

Alternatively, it is however possible also to use other materials for the inner bandage and more particularly for one or both layers of the inner bandage, for example woven fabrics, knitted fabrics or crocheted fabrics, or foams. It is for example possible for the padding layer to be a layer of nonwoven wadding, more particularly a layer of a thermal fusion nonwoven, which may optionally also be pre-needled.

In both processes—thermal bonding and thermal fusion—the fibers of the nonwoven are laid in a particular direction in a combing process and prepared in a textile functionalization process in the form of nonwoven rolls and temperature-stabilized or temperature- and pressure-stabilized for further processing. During the thermal fusion process, fibers having different melting points are fused together by means of hot-air dryers. In the thermal bonding process, the fibers are fused between heated calender rolls by means of heat and pressure. The result in both cases is soft, homogeneous nonwoven fabrics that are ideal and suitable for technical uses. The thermal fusion process is better suited for padding layers because of the absence of pressure.

In order to achieve an optimal padding effect, the thickness of the padding layer of the inner bandage may preferably be 0.3-12 mm, preferably 0.4-6 mm, and further preferably 0.5-3 mm, more preferably 0.6-1.2 mm.

The support layer of the inner bandage may be a thermal bond nonwoven. The thermal bond nonwoven preferably has only low extensibility while at the same time having the desired rigidity.

The nonwoven material of the compression bandage (outer bandage) is likewise preferably a thermal bond nonwoven. For overstitching, the nonwoven material of the respective bandages is fed to a warp knitting machine and overstitched using an elastic stitching thread that may preferably be selected from a group composed of cotton spun crepe threads, cotton twisted crepe threads, textured polyamide yarns, textured polyester yarns, rubber threads or polyurethane elastane threads or a combination thereof and, where there is a plurality of layers, joined together. The material used to produce the compression bandage is a non-elastic thermoplastic, which are normally unsuitable for the production of elastic fabrics since, as a consequence of their spinning process, they tend to have a higher crystalline structure compared to elastomers. The absence of an amorphous structure, which in contrast thereto ensures good extensibility in elastomers.

The stitching thread can alternatively also be referred to as a warp thread. The thread runs in the machine direction of the warp knitting machine and not transversely thereto.

The completely stitched fabric of the inner bandage and/or of the compression bandage always has optimized extension, it being particularly preferable that the maximum extensibility of the inner bandage and/or of the compression bandage, which corresponds to a specified optimal extensibility, and extension over and above this, is limited by a limit of extension. This can significantly increase the security of attachment, since it is possible even for inexperienced users to stretch the inner bandage and/or the compression bandage maximally up to the limit of extension, wherein not only the maximum extensibility, but at the same time also the optimal extension and hence the optimal compression pressure is then achieved and the inner bandage and/or the compression bandage can be applied in this maximally stretched state.

As a result of the overstitching by means of a stitch-bonding process, the inner bandage and/or the compression bandage in the tension-released state, after the stitched fabric has been further processed by means of lengthwise assembly, are set into corrugations with the result that an irregular surface is formed. As a result of this irregular surface, what is also achieved, besides the primary function as a compensation layer and the secondary function of adjustable extensibility and thus increased security of attachment, is that the corrugations give rise to a surface pattern formed from the peaks and troughs in the material that is not completely eliminated even in the maximally stretched state, with the result that a massage or drainage effect is additionally obtained when used in therapy.

The classification into categories of short-, medium- or long-stretch bandages is made according to extensibility and can be found for example in P. Asmussen, B. Sollner, Kompressionstherapie Prinzipien and Praxis [Compression therapy: Principles and practice], Urban & Fischer in Elsevier, 2004, page 121. Extensibilities are here determined in accordance with DIN 61632.

In addition, it may be the case that the compression bandage (second outer bandage) has an extensibility, measured in accordance with DIN 61632, of D_{fix}>90%, in particular of from 40% to 80%.

When applying the compression bandage combination, it is advantageous when the first and second bandages overlap completely and more particularly edge-to-edge and are adhesively joined to one another over the entire extension range of 0 to D_{fix}.

Both bandages are preferably cohesively adhesive in design. A non-slip join can then be provided by virtue of the cohesively adhering second section of the inner bandage and the likewise cohesively adhering outer bandage that interacts therewith.

Cohesive adhesion means that there is no adhesion to, for example, skin or clothing, the adhesion being only between the bandage layers (surface to surface).

The adhesive forces are determined by the method described below:

The adhesive force is the force determined which is needed to part cohesive samples in a test referred to as a 180° T-peel test.

The cohesive coated textile is laid out without tension or wrinkling. A sample 10 cm wide and 40 cm long is cut from it. The 40 cm long sample strip is cut in the middle into two strips 20 cm in length.

The two 20 cm long strips are placed on top of one another so that side A of the first strip is lying on side B of the second strip. The sample thus prepared is placed on a heated (40° C.) stainless steel plate and rolled with a heated (40° C.) metal roller a total of 40 times within a 30 sec period (20 times back and forth).

The weight of the metal roller is 8 kg for a 10 cm sample width, i.e. 0.8 kg per cm sample width for widths other than 10 cm.

The force required to part the layers of the sample is then determined in a force-extension tester (manufacturers: Zwick, Instron). For this, the end of the first layer is clamped in the lower jaw and the other end of the second layer in the upper jaw, ensuring that the sample is as far as possible positioned between the jaws without warpage, i.e. under minimal tension, so that no “resting force” is being applied. This arrangement corresponds to a 180° T-peel test. For the measurement, the jaws move vertically apart from one another and the parting force in action (corresponding to the momentary adhesion of the sample) is recorded continuously. The separation energy is recorded and calculated by integrating the force over the distance traveled by the jaws, and from this energy is recorded and calculated the average parting force=adhesive force in cN/cm. This adhesive force corresponds to the numerical data for the examples.

It is particularly advantageous in this context when the adhesive force of the cohesively adhering bandage section and also of the cohesively adhering second bandage is 20-150 cN/cm, more preferably 30-100 cN/cm and more preferably 40-80 cN/cm.

Preferably, one or both bandages have an open-pored coating of adhesive on one side, giving rise to a cohesive adhesive function from this surface. In the case of the compression bandage (outer bandage), a cohesive coating on both sides is also possible.

The components (bandages) adhere to one another when applied and have a synergistic compression effect. This is understood as meaning the interaction of the compression pressure collectively on the body part. The multilayer dressing is additionally intended to solve the problem of mild ambulatory venous and arterial hypertension when being worn as a permanent dressing.

For the production of the inner bandage, a process for producing a nonwoven-based compression bandage comprising a thermal bond nonwoven layer stitched with a superhydrophilic or superhydrophobic nonwoven layer, serves as an example. The thermal bond nonwoven is fed to a warp knitting machine in an unstretched state together with a waterjet nonwoven, which may also be pre-needled. In the warp knitting machine, the nonwoven (thermal bond) and the superhydrophilic or superhydrophobic nonwoven are stitched with an elastic material so that an elastic composite that can no longer be separated by hand but is nevertheless controllable is obtained. The padding material formed from the completely stitched fabric always also has optimized extension.

Optimized extension is generally the result of incorporating elastic threads (high-twist cotton threads in the form of spun or twisted crepe threads, textured polyamide or polyester yarns, rubber threads or polyurethane-elastane threads) into a non-elastic nonwoven, for example a stiff thermal bond nonwoven. The technique for stitching the nonwoven with elastic material is regulated by the stitch length and tension such that the fibers form a compensation layer (padding layer) on one side of the composite and thus have a high level of skin comfort, with the result that the inner bandage itself has two recognizably different sides that are very functional for pressure compensation. This layer is identical to the padding layer disclosed in DE102015226706.

The invention relates more particularly (component B) to a process for producing a nonwoven-based compression bandage of the invention comprising, for example, an autogenously bonded fiber surface that is consolidated by a chemical agent or in a thermal or mechanical process, referred to as a thermal bond nonwoven layer, and an elastic, non-elastomeric yarn. The thermal bond nonwoven layer is stitched with controlled tensile force in a warp knitting machine using non-elastomeric, heat-shrinkable textile threads in a controlled stretched state. The compressible material formed from the completely stitched fabric always has optimized extension; the optimized extension is the result of incorporating non-elastomeric threads (textured polyamide or polyester yarns) and a non-elastic nonwoven, e.g. rigid thermal bond nonwoven. The technique for stitching the nonwoven with non-elastomeric materials and the stitch length are regulated such that a limited compression pressure of 10 to 30 mmHg is reliably achieved in subsequent use.

The stitched fabric can then be further processed into bandages by means of lengthwise assembly. This bandage can be used as an aid in compression therapy, preferably as a second (outer) layer of a 2-layer compression dressing.

The compression bandage preferably has two recognizable and different sides.

As the second (outer) layer, the bandage regulates the overall contact pressure and ensures the necessary rigidity. The non-elastomeric and elastic threads ensure that a compression bandage composition of the invention does not fall below or exceed a pressure of 20-40 mmHg. This function is not created by competing products.

The invention is described in more detail hereinbelow with reference to a drawing. Further advantages and features of the invention are additionally apparent from the other application documents.

In the drawings:

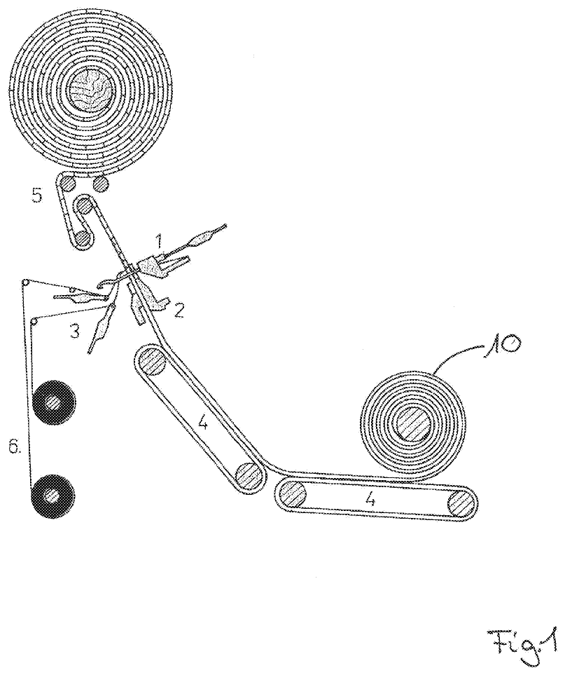

FIG. 1 shows a process for producing a compression bandage of the invention and

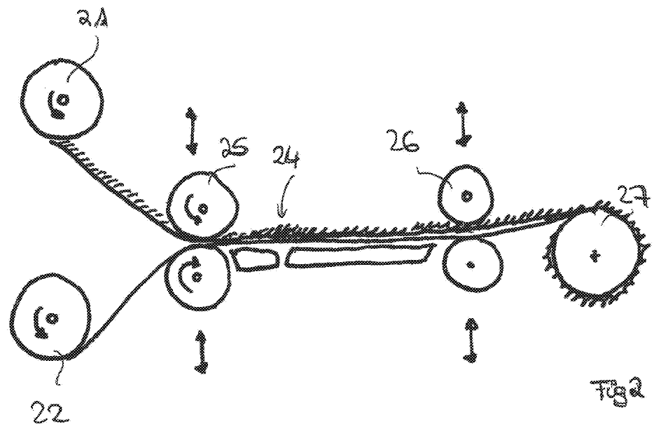

FIG. 2 shows a process for producing a first bandage of a compression bandage combination of the invention.

FIG. 1 shows a process for producing a compression bandage of the invention, said bandage also being capable of serving as an outer bandage for a compression bandage combination of the invention. Here, the reference numeral 10 denotes a nonwoven material to be supplied, more particularly a thermal bond nonwoven material, which is supplied to a stitch-bonding device via conveying devices 4. The reference numerals 1, 2, and 3 indicate the stitch-bonding device, which is a conventional stitch-bonding device for carrying out overstitching by the Maliwatt or Malimo process. For this, elastic stitching threads are fed in via the rollers, which are indicated by 6. The overstitched and thus elasticized nonwoven is rolled up at reference numeral 5. This can then be followed by assembly of a compression bandage of the invention.

A first preferred exemplary embodiment is shown in the table below:

### EXEMPLARY EMBODIMENT OF A COMPRESSION BANDAGE

| | |
|---|---|
| Base nonwoven | Fibrous nonwoven (staple |
| | fiber nonwoven) composed of |
| | cotton, wool, viscose, |
| | polyamide, polyester, |
| | acrylic, polyolefin or |
| | mixtures of said fibers |
| | (mechanically or chemically |
| | bonded) |
| | Or |
| | Spunbond composed of |
| | polyamide, polyester, |
| | polyolefin, acrylic or |
| | mixtures thereof |
| Basis weight/color of base | 20-40 g/m², white or colored |
| nonwoven | |
| Structure of base nonwoven | Smooth or open-pored or |
| | perforated or embossed |
| Stitching thread | 78 dtex to 320 dtex textured |
| | multifilament yarn composed |
| | of polyamide 6, 6.6, |
| | polyester, polyethylene |
| | terephthalate, polybutylene |
| | terephthalate |
| Color of stitching thread | White, colorless/transparent |
| | or colored |
| Stitching thread density | 24 to 96 threads per 10 cm |
| | width |
| Stitching thread stitch | 2.0 to 5.0 mm |
| length | |
| Stitching thread binding | Open fringe or closed fringe |
| | or tricot |
| Basis weight of component B | Uncoated: 25 to 80 g/m² |
| (DIN 61632) | stretched |
| | Coated: 30 to 100 g/m² |
| | stretched |
| Extensibility (DIN 61632 at | 40 to 100% in longitudinal |
| K = 3 N/cm) | direction (MD) |
| | 0 to 50% in transverse |
| | direction (CD) |

FIG. 2 shows firstly how a padding layer 21 composed of nonwoven wadding and a support layer 22 are fed in here as a thermal bond nonwoven layer from a roll material and are joined to one another in a stitch-bonding process by means of a warp knitting machine having the reference numeral 24. The two layers are fixed to one another beforehand via a roller guide 25. The stitch-bonding process is executed using a hook, by means of which the layers are overstitched with the elastic stitching thread. The material joined together via the elastic stitching thread is then passed through a further roller guide 26 and wound onto a roll 27, it being optionally possible for preassembly in the longitudinal direction into bandages to take place. The stitching thread is an elastically pre-stretched thread, which is inserted with a specified stitch length and a specified stitching thread tension, thereby leading, after tension release in the elastic material, to contraction of the layer composite (fabric).

1. A compression bandage comprising a nonwoven-based bandage layer overstitched by elastic textile threads by means of a stitch-bonding process, wherein the textile threads are heat-shrinkable and non-elastomeric and wherein the compression bandage has elastic extensibility after the textile threads have been heat-shrunk by 50-200%.

2. The compression bandage of claim 1, wherein the bandage layer consists of a chemically, thermally and/or mechanically consolidated nonwoven fabric.

3. The compression bandage of claim 1, wherein the nonwoven bandage layer is a nonwoven fabric which is non-elastic or stretch-elastic.

4. The compression bandage of claim 1 wherein the textile threads are textured polyamide yarns and/or textured polyester yarns and/or polyethylene phthalate and/or polybutylene terephthalate or combinations thereof.

5. The compression bandage of claim 1, wherein the nonwoven-based bandage layer is formed from a fibrous nonwoven comprising cotton, wool, viscose, polyamide, polyester, acrylic, polyolefin or mixtures of said fibers.

6. The compression bandage of claim 1, wherein the nonwoven-based bandage layer comprises a spunbond fabric selected from the consisting of polyamide, polyester, polyolefin, acrylic and mixtures thereof.

7. The compression bandage of claim 1, wherein the stitch length of the elastic textile threads in the stitch-bonding process is 2 to 5 mm per revolution with a stitching thread tension of not more than 10 cN.

8. The compression bandage of claim 1, wherein the extensibility of the compression bandage, measured in accordance with DIN 61632, is from 40 to 100% in the longitudinal direction (MD).

9. A compression bandage combination comprising a first, inner bandage (12) and a second, outer bandage which second, outer bandage is the compression bandage of claim 1, wherein the second outer bandage is appliable over the first, inner bandage (12), and wherein the first, inner bandage comprises a first section (L1) that has a padding layer at least on its side facing the skin of a wearer and a cohesively adhering second section (L2).

10. The compression bandage combination of claim 9 wherein the two sections (L1, L2) of the first, inner bandage (12) are joined to one another and adjoin one another in the longitudinal direction of the bandage (12) or completely or partially cover one another.

11. The compression bandage combination of claim 9, wherein the first bandage comprises a first, padding layer (21) and a second, support layer (22), the two layers (21, 22) being joined to one another in the unstretched state by means of a stitch-bonding process via an elastic stitching thread, the stitch length being 1.5 to 3 mm/rev with a stitching thread tension of not more than 4 cN.

12. The compression bandage combination of claim 9, wherein the first, inner bandage comprises a first, padding layer (21) and a second support layer (22) and the material of one or both of the layers (21, 22) of the first, inner bandage is non-elastic.

13. The compression bandage combination of claim 9, wherein the first, inner bandage comprises a first, padding layer (21) and a second support layer (22) and at least one of the two layers (21, 22)—the padding layer (21) and the support layer (22)—are composed of a nonwoven material.

14. The compression bandage combination of claim 9, wherein the first, inner bandage comprises a first, padding layer (21) and a second support layer (22) and wherein the padding layer (21) is a thermal fusion nonwoven.

15. The compression bandage combination of claim 9 wherein the first, inner bandage and second outer bandage overlap completely.

16. A process for producing a compression bandage as claimed in claim 1, comprising overstitching a nonwoven-based bandage layer with elastic textile threads in a stitch-bonding process, and heat treating the overstitched bandage layer to result in shrinkage of the textile threads of between 50 and 200.

17. A process of treating any one of venous disorders, chronic venous insufficiency, and venous leg ulcers optionally in the presence of concomitant peripheral arterial occlusive disease comprising utilizing the compression bandage of claim 1.

18. The compression bandage of claim 1, wherein the compression bandage has elastic extensibility after the textile threads have been heat-shrunk by 50% to 90%.

19. The compression bandage of claim 1, wherein the compression bandage has elastic extensibility after the textile threads have been heat-shrunk by 50% to 70%.

20. The compression bandage combination of claim 15, wherein the first, inner bandage and second outer bandage overlap edge-to-edge and are adhesively joined to one another over the entire extension range of 0 to D_{fix}.
